(19) United States
(12) Patent Application Publication
(10) Pub. No.: US 2023/0295658 A1
(43) Pub. Date: Sep. 21, 2023

(54) COMPOSITIONS AND METHODS FOR IN VIVO GENE TRANSFER

(71) Applicant: The Children's Hospital of Philadelphia, Philadelphia, PA (US)

(72) Inventors: Stefano Rivella, Philadelphia, PA (US); Perry Demsko, Lansdale, PA (US)

(21) Appl. No.: 18/033,627

(22) PCT Filed: Nov. 10, 2021
(86) PCT No.: PCT/US21/58764

§ 371 Date: Apr. 25, 2023

Related U.S. Application Data
(60) Provisional application No. 63/111,899, filed on Nov. 10, 2020.

(51) Int. Cl. C12N 15/86; C12N 15/113; A61K 31/7088; A61K 31/395; A61P 1/16

(52) U.S. Cl. CPC C12N 15/86; C12N 15/1138; A61K 31/7088; A61K 31/395; A61P 1/16; C12N 2750/14143; C12N 2760/20243; C12N 2310/11; C12N 2320/31; A61K 48/00

(57) ABSTRACT

Methods and compositions for improved gene therapy are disclosed.

This application claims priority under 35 U.S.C. § 119(e) to U.S. Provisional Patent Application No. 63/111,899, filed Nov. 10, 2020. The foregoing application is incorporated by reference herein.

### FIELD OF THE INVENTION

The present invention relates to the field of gene therapy. More specifically, the invention provides compositions and methods for improved gene therapy.

### BACKGROUND OF THE INVENTION

Several publications and patent documents are cited throughout the specification in order to describe the state of the art to which this invention pertains. Each of these citations is incorporated herein by reference as though set forth in full.

The current paradigm for gene therapies—such as gene therapies directed at metabolic storage diseases, deficiencies in immunity, hemoglobinopathies, and the like—involves the ex vivo transduction of patient cells, primarily hematopoietic stem cells (HSCs). This process involves the collection, sorting, and selection of a patient's cells outside the body in an expensive, inefficient, and labor-intensive process. Additionally, prior to reinfusion of the corrected cells, a pre-conditioning regimen is often required wherein chemical, radiological, or other means are used to ablate either wholly or partially the resident cell population to allow for the corrected cells to have a niche to populate. These pre-conditioning regimens have many undesirable side-effects and comorbidities including an increased chance of cancer and severely reduced or even eliminated immune function until the therapeutic cells have reconstituted their niche. Additionally, ex vivo handling of certain cell types such as HSCs can eliminate their stemness or self-renewal capability, reducing the effectiveness of the therapy. Thus, there is an ongoing and unmet need for improved compositions and methods for gene therapy.

### SUMMARY OF THE INVENTION

In accordance with one aspect of the instant invention, methods for increasing gene transfer to cells other than the liver and/or decreasing liver toxicity are provided. In certain embodiments, the methods are performed before, after, and/or at the same time as a gene therapy. In a particular embodiment, the gene therapy vector is an AAV or VSV-G vector. In a particular embodiment, the method comprises reducing expression and/or blocking AAVR or LDL-R in the liver, such as by administering an AAVR or LDL-R inhibitor, particularly to the liver. In certain embodiments, AAVR or LDL-R are inhibited prior to, after, and/or at the same time as a gene therapy vector, particularly at least before the gene therapy vector. In a particular embodiment, the inhibitor is an inhibitory nucleic acid molecule or a nucleic acid molecule encoding the inhibitory nucleic acid molecule. In certain embodiments, the inhibitory nucleic acid molecule regulates gene expression by RNA interference (RNAi). Examples of inhibitory nucleic acid molecules include antisense oligonucleotides, miRNA, siRNA, and shRNA. In a particular embodiment, the method further comprises administering a HSC mobilization agent such as plerixafor to the subject. In a particular embodiment, the gene therapy vector comprises CD47.

In accordance with another aspect of the instant invention, variant viral envelope proteins, particularly variant VSV-G, are provided.

### BRIEF DESCRIPTIONS OF THE DRAWINGS

FIG. 1 provides a graph of the expression of LDLR in WT mice and mice treated with a scrambled antisense oligonucleotide (ASO) or an anti-LDLR antisense oligonucleotide.

### DETAILED DESCRIPTION OF THE INVENTION

In vivo gene therapy, or gene therapy delivered into the body, would reduce or eliminate most of the difficulties seen with the present ex vivo paradigm. At the most ideal, in vivo gene therapy would involve a simple injection of vector into the patient's body, into the blood and/or other tissues depending on the application. In vivo gene therapy would eliminate the ex vivo handling of patient cells and potentially obviate preconditioning regimens. Presently, the primary vectors used to accomplish integrative gene therapies, including the ex vivo examples above, are adenoviral associated viral vectors (AAV) using a variety of envelopes, as well as lentivirus pseudotyped with the G-protein of vesicular stomatitis virus G (VSV-G) as the primary envelope.

In accordance with one aspect of the present invention, methods for increasing AAV gene transfer outside of the liver and/or decreasing AAV liver toxicity (e.g., that associated with gene therapy) are provided. For AAV, gene transfer in vivo is well established. However, most of the AAV viruses have a liver tropism and target the AAV receptor (AAVR). Other tissues are targeted by these viruses, but most of the time at lower efficiency. In many cases, tissues other than liver, are the correct target to cure a specific disease by gene therapy. Increasing the number of viral particles infused can increase the chances to deliver the transgene to other tissues. However, this also leads to liver toxicity. Therefore, decreasing and/or preventing infection of the liver would increase the delivery of the transgene to other tissues and prevent liver complications and toxicity. Reducing expression of the AAVR in the liver can retarget AAV vectors to other tissues (including hematopoietic stem cells) with normal or super physiological levels, with increased therapeutic potential and less organ toxicity.

The methods comprise the use of an AAVR inhibitor such as inhibitory nucleic acid molecules—such as antisense oligonucleotides, siRNA, miRNA, or shRNA—directed against AAVR and/or drugs/compounds (e.g., liver specific drugs) that specifically target AAVR, particularly in the liver. The reduction in AAVR expression or blocking of AAVR in the liver will allow AAV vectors (e.g., therapeutic AAV vectors for gene therapy) to deliver their cargo to other tissues more efficiently. Examples of AAVR inhibitors include, without limitation, proteins, polypeptides, peptides, antibodies, small molecules, and nucleic acid molecules. In a particular embodiment, the AAVR inhibitor is an inhibitory nucleic acid molecule, such as an antisense, siRNA, miRNA, or shRNA molecule (or a nucleic acid molecule encoding the inhibitory nucleic acid molecule).

In a particular embodiment, the methods comprise administering at least one AAVR inhibitor and one AAV vector, particularly an AAV gene therapy, to a patient. The AAVR inhibitor may be administered consecutively and/or simultaneously with the AAV vector or AAV gene therapy. In a particular embodiment, the AAVR inhibitor is administered to the liver. In a particular embodiment, the method further comprises administering a HSC mobilization agent such as plerixafor to the subject. HSC are generally hidden in the bone marrow, mobilization of these cells will increase the exposure of these cells to AAV vectors administered in vivo. In a particular embodiment, the HSC mobilization agent is administered after and/or simultaneously with suppression of the expression of AAVR in the liver.

When an inhibitory nucleic acid molecule (e.g., an shRNA, miRNA, siRNA, or antisense) is delivered to a cell or subject, the inhibitory nucleic acid molecule may be administered directly or an expression vector may be used. In a particular embodiment, the inhibitory nucleic acid is administered directly. In a particular embodiment, the inhibitory nucleic acid molecules are delivered (e.g., via infection, transfection, electroporation, etc.) and expressed in cells via a vector (e.g., a plasmid), particularly a viral vector. The expression vectors of the instant invention may employ a strong promoter, a constitutive promoter, and/or a regulated promoter. In a particular embodiment, the inhibitory nucleic acid molecules are expressed transiently. In a particular embodiment, the promoter is cell-type specific (e.g., liver cells). Examples of promoters are well known in the art and include, but are not limited to, RNA polymerase II promoters, the T7 RNA polymerase promoter, and RNA polymerase III promoters (e.g., U6 and H1; see, e.g., Myslinski et al. (2001) Nucl. Acids Res., 29:2502-09). Examples of expression vectors for expressing the molecules of the invention include, without limitation, plasmids and viral vectors (e.g., adeno-associated viruses (AAVs), adenoviruses, retroviruses, and lentiviruses).

Compositions comprising at least one AAVR inhibitor and at least one carrier (e.g., a pharmaceutically acceptable carrier) are also encompassed by the instant invention. Except insofar as any conventional carrier is incompatible with the variant to be administered, its use in the pharmaceutical composition is contemplated. In a particular embodiment, the carrier is a pharmaceutically acceptable carrier for intravenous administration or injection into the bloodstream.

Adeno-associated virus receptor (AAVR) is a glycosylated protein containing five polycystic kidney disease (PKD) repeat domains in its extracellular portion and is a key proteinaceous receptor for multiple AAV serotypes for viral entry (Ibraghimov-Beskrovnaya, et al. (2000) Hum. Mol. Genet. (2000) 9:1641-1649; Pillay, et al. (2016) Nature 530:108-112; Pillay, et al. (2017) J. Virol., 91:e00391-17; Zhang, et al. (2019) Nature Microbiol., 4: 675-682; Zhang et al. (2019) Nature Comm., 10: 3760; Zengel et al. (2020) Adv. Virus Res., 106:39-84). AAVR was previously known as type I transmembrane protein KIAA0319L (Pillay, et al. (2016) Nature 530:108-112). In a particular embodiment, the AAVR is human. Examples of amino acid and nucleotide sequences of AAVR are provided in Gene ID: 79932 and GenBank Accession Nos: NM_024874.5 and NP_079150.3. An example of a nucleotide sequence encoding AAVR is (SEQ ID NO: 1):

| | | | | | | |
|---|---|---|---|---|---|---|
| 1 | gtttccggcc | gccgtcgctg | tccagggagg | ctgaggcgag | aggtagctgt | ccgggtgggg |
| 61 | agcccgcact | accttcttcc | tcttcctcct | cctcctccgg | gtgaggggag | cgaaggttgg |
| 121 | gggtccccga | gcccatggac | caggaggagg | cggaggccgc | cgagagccgg | ggccccgcta |
| 181 | tgtggccctg | agccccgtgt | actggttctg | cctgtctgga | gggccatgga | gaagaggctg |
| 241 | ggagtcaagc | caaatcctgc | ttcctggatt | ttatcaggat | attattggca | gacatctgcg |
| 301 | aagtggttga | gaagcctgta | cctgttttat | acttgctttt | gcttcagcgt | tctgtggttg |
| 361 | tcaacagatg | ccagtgagag | caggtgccag | caggggaaga | cacaatttgg | agttggcctg |
| 421 | agatctgggg | gagaaaatca | cctctggctt | cttgaaggaa | ccccctctct | ccagtcatgt |
| 481 | tgggctgcct | gctgccagga | ctctgcctgc | catgtctttt | ggtggctaga | agggatgtgc |
| 541 | attcaggcag | actgcagcag | gccccagagc | tgccgggctt | ttaggacaca | ctcctccaat |
| 601 | tccatgctgg | tgtttttaaa | aaaattccaa | actgcagatg | atttgggctt | tctacctgaa |
| 661 | gatgatgtac | cacatcttct | ggggctaggt | tggaactggg | catcttggag | gcagagccca |
| 721 | cccagagctg | cactcagacc | tgctgtatct | tccagtgacc | agcagagctt | aatcaggaag |
| 781 | cttcagaaga | gaggtagtcc | cagtgacgta | gttacaccta | tagtgacaca | gcattctaaa |
| 841 | gtgaatgact | ccaacgaatt | aggtggtctg | actaccagtg | gctctgcaga | ggtccacaag |
| 901 | gcgattacaa | tttccagtcc | cctaaccaca | gacctgactg | cagagctgtc | tggtgggcca |
| 961 | aagaatgtat | cagtgcaacc | tgaaatatca | gagggtcttg | ctactacgcc | cagcactcaa |
| 1021 | caagtaaaaa | gttctgagaa | aacccagatt | gctgtccccc | agccagtggc | tccctcctac |
| 1081 | agttatgcta | cccctacccc | ccaggcctct | ttccagagca | cctcagcacc | atacccagtt |
| 1141 | ataaaggaac | tggtggtatc | tgctggagag | agtgtccaga | taaccctgcc | taagaatgaa |
| 1201 | gttcaattaa | atgcatatgt | tctccaagaa | ccacctaaag | gagaaaccta | cacctacgac |
| 1261 | tggcagctga | ttactcatcc | tagagactac | agtggagaaa | tggaagggaa | acattcccag |
| 1321 | atcctcaaac | tatcgaagct | cactccaggc | ctgtatgaat | tcaaagtgat | tgtagagggt |
| 1381 | caaaatgccc | atggggaagg | ctatgtgaac | gtgacagtca | agccagagcc | ccgtaagaat |
| 1441 | cggcccccca | ttgctattgt | gtcacctcag | ttccaggaga | tctctttgcc | aaccacttct |
| 1501 | acagtcattg | atggcagtca | aagcactgat | gatgataaaa | tcgttcagta | ccattgggaa |
| 1561 | gaacttaagg | ggcctctaag | agaagagaag | atttctgaag | atacagccat | attaaaacta |
| 1621 | agtaaactcg | tccctgggaa | ctacactttc | agcttgactg | tagtagactc | tgatggagct |
| 1681 | accaactcta | ctactgcaaa | cctgacagtg | aacaaagctg | tggattaccc | ccctgtggcc |
| 1741 | aacgcaggcc | ccaaccaagt | gatcaccctg | ccccaaaact | ccatcaccct | ctttgggaac |
| 1801 | cagagcactg | atgatcatgg | catcaccagc | tatgagtggt | cactcagccc | aagcagcaaa |
| 1861 | gggaaagtgg | tggagatgca | gggtgttaga | acaccaacct | tacagctctc | tgcgatgcaa |
| 1921 | gaaggagact | acacttacca | gctcacagtg | actgacacaa | taggacagca | ggccactgct |
| 1981 | caagtgactg | ttattgtgca | acctgaaaac | aataagcctc | ctcaggcaga | tgcaggccca |
| 2041 | gataaagagc | tgacccttcc | tgtggatagc | acaaccctgg | atggcagcaa | gagctcagat |
| 2101 | gatcagaaaa | ttatctcata | tctctgggaa | aaaacacagg | gacctgatgg | ggtgcagctc |
| 2161 | gagaatgcta | acagcagtgt | tgctactgtg | actgggctgc | aagtggggac | ctatgtgttc |
| 2221 | accttgactg | tcaaagatga | gaggaacctg | caaagccaga | gctctgtgaa | tgtcattgtc |
| 2281 | aaagaagaaa | taaacaaacc | acctatagcc | aagataactg | ggaatgtggt | gattacccta |
| 2341 | cccacgagca | cagcagagct | ggatggctct | aagtcctcag | atgacaaggg | aatagtcagc |
| 2401 | tacctctgga | ctcgagatga | ggggagccca | gcagcagggg | aggtgttaaa | tcactctgac |
| 2461 | catcacccta | tcctttttct | ttcaaacctg | gttgagggaa | cctacacttt | tcacctgaaa |
| 2521 | gtgaccgatg | caaagggtga | gagtgacaca | gaccggacca | ctgtggaggt | gaaacctgat |
| 2581 | cccaggaaaa | acaacctggt | ggagatcatc | ttggatatca | acgtcagtca | gctaactgag |
| 2641 | aggctgaagg | ggatgttcat | ccgccagatt | ggggtcctcc | tgggggtgct | ggattccgac |
| 2701 | atcattgtgc | aaaagattca | gccgtacacg | gagcagagca | ccaaaatggt | attttttgtt |
| 2761 | caaaacgagc | ctccccacca | gatcttcaaa | ggccatgagg | tggcagcgat | gctcaagagt |
| 2821 | gagctgcgga | agcaaaaggc | agactttttg | atattcagag | ccttggaagt | caacactgtc |
| 2881 | acatgtcagc | tgaactgttc | cgaccatggc | cactgtgact | cgttcaccaa | acgctgtatc |
| 2941 | tgtgaccctt | tttggatgga | gaatttcatc | aaggtgcagc | tgagggatgg | agacagcaac |
| 3001 | tgtgagtgga | gcgtgttata | tgttatcatt | gctacctttg | tcattgttgt | tgccttggga |
| 3061 | atcctgtctt | ggactgtgat | ctgttgttgt | aagaggcaaa | aaggaaaacc | caagaggaaa |
| 3121 | agcaagtaca | agatcctgga | tgccacggat | caggaaagcc | tggagctgaa | gccaacctcc |
| 3181 | cgagcaggca | tcaaacagaa | aggccttttg | ctaagtagca | gcctgatgca | ctccgagtca |
| 3241 | gagctggaca | gcgatgatgc | catctttaca | tggccagacc | gagagaaggg | caaactcctg |
| 3301 | catggtcaga | atggctctgt | acccaacggg | cagacccctc | tgaaggccag | gagcccgcgg |
| 3361 | gaggagatcc | tgtagccacc | tggtctgtct | cctcagggca | gggcccagca | cactgcccgg |
| 3421 | ccagtcctcc | tacctcccga | gtctgcgggc | agctgctgtc | ccagcatctg | ctggtcattt |
| 3481 | cgccctgaca | gtcccaacca | gaacccctgg | gacttgaatc | cagagacgtc | ctccaggaac |
| 3541 | ccctcaacga | agctgtgaat | gaagaggttt | cctctttaaa | cctgtctggt | gggcccccag |
| 3601 | atatcctcac | ctcagggcct | cctttttttg | caaactcctc | ccctcccccg | agggcagacc |
| 3661 | cagccagctg | ctaagctctg | cagctcccca | gtggacagtg | tcattgtgcc | cagagtgctg |
| 3721 | caaggtgagg | cctgctgtgc | tgcccgcaca | cctgagtgca | aaaccaagca | ctgtgggcat |
| 3781 | ggtgtttccc | tctctggggt | agagtacgcc | ctctcgctgg | gcaaagagga | agtggcaccc |
| 3841 | ctcccctcac | cacagatgct | gagatggtag | catagaaatg | atggccgggc | gcggtggctc |
| 3901 | acgcctgtaa | tcccagcact | ttgggaggcc | gaggcgggcg | gatcatgagg | tcaggagatc |
| 3961 | aagaccaccc | tggctaacac | ggtgaaaccc | catctctact | aaaaataaaa | aaaaaaatta |
| 4021 | gccgggtttg | gtggcgtatg | cctgtaatcc | cagctactcg | ggaggctgag | gcaggagaat |
| 4081 | tgcttaaacc | tgggaggtgg | aggctgcagt | gagccaagat | cgtgccactg | cactccagcc |
| 4141 | tgagtgacag | agcaagactc | cgtcaaaaaa | aaaaaaaaaa | aaaaagaaat | gatatctggc |
| 4201 | ccccccttaa | cactggagcc | ccactccctt | ctcccatccg | gcccgagatt | agggaggatt |
| 4261 | gactgtgtca | gggatggcgg | gggcctctc | tcgctgccag | ggcccttgtc | agagcagcca |
| 4321 | ggctggacag | acggcctccc | tcctctccat | ctgaccggca | cctgctgctt | cggggcttag |
| 4381 | gccaccgctc | cctgtcccca | gaggagatag | ccccagatgg | actggaatgt | tgtggcatga |
| 4441 | gagcgcatgt | gtgcgatggc | cccgctgtgg | tcccctctct | gtccctccat | ctgtatgtgt |
| 4501 | tctgtgtccc | ttgcatgtgt | gcgtgttaga | gtgagcgcgt | atgcatcaac | tcattgggct |
| 4561 | cttggctgct | cacaaggcaa | atttgacttg | gaaagacttt | catctccttg | gaaccaagac |
| 4621 | ttcctgagtc | cccctcaccc | tggccctgtt | ccaccatggt | tatctgggta | ttggggaatg |
| 4681 | gaaactttgg | gggagtgact | ttttaaagag | acacttataa | tttctactac | tgcactactg |
| 4741 | tccattgtgg | gatgattaaa | catggtattt | aactgtg | | |

In a particular embodiment, the nucleotide sequence encoding AAVR comprises nucleotides 226-3375 of SEQ ID NO: 1. In certain embodiments, the inhibitory nucleic acid molecule targets a region within nucleotides 226-3375 of SEQ ID NO: 1.

An example of an amino acid sequence of AAVR is (SEQ ID NO: 2):

| | | | | | | |
|---|---|---|---|---|---|---|
| 1 | MEKRLGVKPN | PASWILSGYY | WQTSAKWLRS | LYLFYTCFCF | SVLWLSTDAS | ESRCQQGKTQ |
| 61 | FGVGLRSGGE | NHLWLLEGTP | SLQSCWAACC | QDSACHVFWW | LEGMCIQADC | SRPQSCRAFR |
| 121 | THSSNSMLVF | LKKFQTADDL | GFLPEDDVPH | LLGLGWNWAS | WRQSPPRAAL | RPAVSSSDQQ |
| 181 | SLIRKLQKRG | SPSDVVTPIV | TQHSKVNDSN | ELGGLTTSGS | AEVHKAITIS | SPLTTDLTAE |
| 241 | LSGGPKNVSV | QPEISEGLAT | TPSTQQVKSS | EKTQIAVPQP | VAPSYSYATP | TPQASFQSTS |
| 301 | APYPVIKELV | VSAGESVQIT | LPKNEVQLNA | YVLQEPPKGE | TYTYDWQLIT | HPRDYSGEME |
| 361 | GKHSQILKLS | KLTPGLYEFK | VIVEGQNAHG | EGYVNVTVKP | EPRKNRPPIA | IVSPQFQEIS |
| 421 | LPTTSTVIDG | SQSTDDDKIV | QYHWEELKGP | LREEKISEDT | AILKLSKLVP | GNYTFSLTVV |
| 481 | DSDGATNSTT | ANLTVNKAVD | YPPVANAGPN | QVITLPQNSI | TLFGNQSTDD | HGITSYEWSL |
| 541 | SPSSKGKVVE | MQGVRTPTLQ | LSAMQEGDYT | YQLTVTDTIG | QQATAQVTVI | VQPENNKPPQ |
| 601 | ADAGPDKELT | LPVDSTTLDG | SKSSDDQKII | SYLWEKTQGP | DGVQLENANS | SVATVTGLQV |
| 661 | GTYVFTLTVK | DERNLQSQSS | VNVIVKEEIN | KPPIAKITGN | VVITLPTSTA | ELDGSKSSDD |
| 721 | KGIVSYLWTR | DEGSPAAGEV | LNHSDHHPIL | FLSNLVEGTY | TFHLKVTDAK | GESDTDRTTV |
| 781 | EVKPDPRKNN | LVEIILDINV | SQLTERLKGM | FIRQIGVLLG | VLDSDIIVQK | IQPYTEQSTK |
| 841 | MVFFVQNEPP | HQIFKGHEVA | AMLKSELRKQ | KADFLIFRAL | EVNTVTCQLN | CSDHGHCDSF |
| 901 | TKRCICDPFW | MENFIKVQLR | DGDSNCEWSV | LYVIIATFVI | VVALGILSWT | VICCCKRQKG |
| 961 | KPKRKSKYKI | LDATDQESLE | LKPTSRAGIK | QKGLLLSSSL | MHSESELDSD | DAIFTWPDRE |
| 1021 | KGKLLHGQNG | SVPNGQTPLK | ARSPREEIL | | | |

In a particular embodiment, the AAVR is the mature form. In a particular embodiment, the AAVR has at least 80%, 85%, 90%, 95%, 97%, 99%, or 100% identity, particularly at least 97%, or 99% identity, with one of the above sequences.

In accordance with another aspect of the present invention, methods for increasing vesicular stomatitis virus glycoprotein G (VSV-G) gene transfer outside of the liver and/or decreasing VSV-G liver toxicity are provided. For lentiviral vectors, VSV-G is a multi-functional protein that facilitates both viral targeting to the low-density lipoprotein-receptor (LDL-R) on the cellular surface and pH-dependent viral entry into the cell once the receptor is internalized (Finkleshtein, et al. (2013) PNAS 110 (18):7306-7311; Nikolic, et al. (2018) Nature Comm., 9:1029). The VSV-G is also expressed in the liver at high level. Therefore, decreasing and/or preventing infection of the liver would increase the delivery of the transgene to other tissues and prevent liver complications and toxicity. Reducing expression of the VSV-G in the liver can retarget VSV-G vectors (e.g., VSV-G lentiviral vectors) to other tissues (including hematopoietic stem cells) with normal or super physiological levels, with increased therapeutic potential and less organ toxicity.

The methods comprise the use of an LDL-R inhibitor such as inhibitory nucleic acid molecules—such as antisense oligonucleotides, siRNA, miRNA, or shRNA—directed against LDL-R and/or drugs/compounds (e.g., liver specific drugs) that specifically target LDL-R, particularly in the liver. The reduction in LDL-R expression or blocking of LDL-R in the liver will allow VSV-G vectors (e.g., therapeutic VSV-G vectors or gene therapy) to deliver their cargo to other tissues more efficiently. Examples of LDL-R inhibitors include, without limitation, proteins, polypeptides, peptides, antibodies, small molecules, and nucleic acid molecules. In a particular embodiment, the LDL-R inhibitor is an inhibitory nucleic acid molecule, such as an antisense, siRNA, miRNA, or shRNA molecule (or a nucleic acid molecule encoding the inhibitory nucleic acid molecule).

In a particular embodiment, the methods comprise administering at least one LDL-R inhibitor and one VSV-G vector, particularly a VSV-G gene therapy, to a patient. The LDL-R inhibitor may be administered consecutively and/or simultaneously with the VSV-G vector or VSV-G gene therapy. In a particular embodiment, the LDL-R inhibitor is administered to the liver. In a particular embodiment, the method further comprises administering a HSC mobilization agent such as plerixafor to the subject. HSC are generally hidden in the bone marrow, mobilization of these cells will increase the exposure of these cells to VSV-G vectors administered in vivo. In a particular embodiment, the HSC mobilization agent is administered after and/or consecutively with suppression of the expression of LDL-R in the liver.

When an inhibitory nucleic acid molecule (e.g., an shRNA, siRNA, miRNA, or antisense) is delivered to a cell or subject, the inhibitory nucleic acid molecule may be administered directly or an expression vector may be used. In a particular embodiment, the inhibitory nucleic acid molecule is administered directly. In a particular embodiment, the inhibitory nucleic acid molecules are delivered (e.g., via infection, transfection, electroporation, etc.) and expressed in cells via a vector (e.g., a plasmid), particularly a viral vector. The expression vectors of the instant invention may employ a strong promoter, a constitutive promoter, and/or a regulated promoter. In a particular embodiment, the inhibitory nucleic acid molecules are expressed transiently. In a particular embodiment, the promoter is cell-type specific (e.g., liver cells). Examples of promoters are well known in the art and include, but are not limited to, RNA polymerase II promoters, the T7 RNA polymerase promoter, and RNA polymerase III promoters (e.g., U6 and H1; see, e.g., Myslinski et al. (2001) Nucl. Acids Res., 29:2502-09). Examples of expression vectors for expressing the molecules of the invention include, without limitation, plasmids and viral vectors (e.g., adeno-associated viruses (AAVs), adenoviruses, retroviruses, and lentiviruses).

Compositions comprising at least one LDL-R inhibitor and at least one carrier (e.g., a pharmaceutically acceptable carrier) are also encompassed by the instant invention. Except insofar as any conventional carrier is incompatible with the variant to be administered, its use in the pharmaceutical composition is contemplated. In a particular embodiment, the carrier is a pharmaceutically acceptable carrier for intravenous administration.

Examples of amino acid and nucleotide sequences of LDL-R are provided in Gene ID: 3949 and GenBank Accession Nos: NM_000527.5 and NP_000518.1. An example of a nucleotide sequence encoding LDL-R is (SEQ ID NO: 3):

| | | | | | | |
|---|---|---|---|---|---|---|
| 1 | gtgcaatcgc | gggaagccag | ggtttccagc | taggacacag | caggtcgtga | tccgggtcgg |
| 61 | gacactgcct | ggcagaggct | gcgagcatgg | ggccctgggg | ctggaaattg | cgctggaccg |
| 121 | tcgccttgct | cctcgccgcg | gcggggactg | cagtgggcga | cagatgcgaa | agaaacgagt |
| 181 | tccagtgcca | agacgggaaa | tgcatctcct | acaagtgggt | ctgcgatggc | agcgctgagt |
| 241 | gccaggatgg | ctctgatgag | tcccaggaga | cgtgcttgtc | tgtcacctgc | aaatccgggg |
| 301 | acttcagctg | tgggggccgt | gtcaaccgct | gcattcctca | gttctggagg | tgcgatggcc |
| 361 | aagtggactg | cgacaacggc | tcagacgagc | aaggctgtcc | ccccaagacg | tgctcccagg |
| 421 | acgagtttcg | ctgccacgat | gggaagtgca | tctctcggca | gttcgtctgt | gactcagacc |
| 481 | gggactgctt | ggacggctca | gacgaggcct | cctgcccggt | gctcacctgt | ggtcccgcca |
| 541 | gcttccagtg | caacagctcc | acctgcatcc | cccagctgtg | ggcctgcgac | aacgaccccg |
| 601 | actgcgaaga | tggctcggat | gagtggccgc | agcgctgtag | gggtctttac | gtgttccaag |
| 661 | gggacagtag | cccctgctcg | gccttcgagt | tccactgcct | aagtggcgag | tgcatccact |
| 721 | ccagctggcg | ctgtgatggt | ggccccgact | gcaaggacaa | atctgacgag | gaaaactgcg |
| 781 | ctgtggccac | ctgtcgccct | gacgaattcc | agtgctctga | tggaaactgc | atccatggca |
| 841 | gccggcagtg | tgaccgggaa | tatgactgca | aggacatgag | cgatgaagtt | ggctgcgtta |
| 901 | atgtgacact | ctgcgaggga | cccaacaagt | tcaagtgtca | cagcggcgaa | tgcatcaccc |
| 961 | tggacaaagt | ctgcaacatg | gctagagact | gccgggactg | gtcagatgaa | cccatcaaag |
| 1021 | agtgcgggac | caacgaatgc | ttggacaaca | acggcggctg | ttcccacgtc | tgcaatgacc |
| 1081 | ttaagatcgg | ctacgagtgc | ctgtgccccg | acggcttcca | gctggtggcc | cagcgaagat |
| 1141 | gcgaagatat | cgatgagtgt | caggatcccg | acacctgcag | ccagctctgc | gtgaacctgg |
| 1201 | agggtggcta | caagtgccag | tgtgaggaag | gcttccagct | ggacccccac | acgaaggcct |
| 1261 | gcaaggctgt | gggctccatc | gcctacctct | tcttcaccaa | ccggcacgag | gtcaggaaga |
| 1321 | tgacgctgga | ccggagcgag | tacaccagcc | tcatccccaa | cctgaggaac | gtggtcgctc |
| 1381 | tggacacgga | ggtggccagc | aatagaatct | actggtctga | cctgtcccag | agaatgatct |
| 1441 | gcagcaccca | gcttgacaga | gcccacggcg | tctcttccta | tgacaccgtc | atcagcagag |
| 1501 | acatccaggc | ccccgacggg | ctggctgtgg | actggatcca | cagcaacatc | tactggaccg |
| 1561 | actctgtcct | gggcactgtc | tctgttgcgg | ataccaaggg | cgtgaagagg | aaaacgttat |
| 1621 | tcagggagaa | cggctccaag | ccaagggcca | tcgtggtgga | tcctgttcat | ggcttcatgt |
| 1681 | actggactga | ctggggaact | cccgccaaga | tcaagaaagg | gggcctgaat | ggtgtggaca |
| 1741 | tctactcgct | ggtgactgaa | aacattcagt | ggcccaatgg | catcacccta | gatctcctca |
| 1801 | gtggccgcct | ctactgggtt | gactccaaac | ttcactccat | ctcaagcatc | gatgtcaacg |
| 1861 | ggggcaaccg | gaagaccatc | ttggaggatg | aaaagaggct | ggcccacccc | ttctccttgg |
| 1921 | ccgtctttga | ggacaaagta | ttttggacag | atatcatcaa | cgaagccatt | ttcagtgcca |
| 1981 | accgcctcac | aggttccgat | gtcaacttgt | tggctgaaaa | cctactgtcc | ccagaggata |
| 2041 | tggttctctt | ccacaacctc | acccagccaa | gaggagtgaa | ctggtgtgag | aggaccaccc |
| 2101 | tgagcaatgg | cggctgccag | tatctgtgcc | tccctgcccc | gcagatcaac | ccccactcgc |
| 2161 | ccaagtttac | ctgcgcctgc | ccggacggca | tgctgctggc | cagggacatg | aggagctgcc |
| 2221 | tcacagaggc | tgaggctgca | gtggccaccc | aggagacatc | caccgtcagg | ctaaaggtca |
| 2281 | gctccacagc | cgtaaggaca | cagcacacaa | ccacccgacc | tgttcccgac | acctcccggc |
| 2341 | tgcctggggc | cacccctggg | ctcaccacgg | tggagatagt | gacaatgtct | caccaagctc |
| 2401 | tgggcgacgt | tgctggcaga | ggaaatgaga | agaagcccag | tagcgtgagg | gctctgtcca |
| 2461 | ttgtcctccc | catcgtgctc | ctcgtcttcc | tttgcctggg | ggtcttcctt | ctatggaaga |
| 2521 | actggcggct | taagaacatc | aacagcatca | actttgacaa | ccccgtctat | cagaagacca |
| 2581 | cagaggatga | ggtccacatt | tgccacaacc | aggacggcta | cagctacccc | tcgagacaga |
| 2641 | tggtcagtct | ggaggatgac | gtggcgtgaa | catctgcctg | gagtcccgtc | cctgcccaga |
| 2701 | acccttcctg | agacctcgcc | ggccttgttt | tattcaaaga | cagagaagac | caaagcattg |
| 2761 | cctgccagag | ctttgtttta | tatatttatt | catctgggag | gcagaacagg | cttcggacag |
| 2821 | tgcccatgca | atggcttggg | ttgggatttt | ggtttcttcc | tttcctcgtg | aaggataaga |
| 2881 | gaaacaggcc | cggggggacc | aggatgacac | ctccatttct | ctccaggaag | ttttgagttt |
| 2941 | ctctccaccg | tgacacaatc | ctcaaacatg | gaagatgaaa | ggggagggga | tgtcaggccc |
| 3001 | agagaagcaa | gtggctttca | acacacaaca | gcagatggca | ccaacgggac | cccctggccc |
| 3061 | tgcctcatcc | accaatctct | aagccaaacc | cctaaactca | ggagtcaacg | tgtttacctc |
| 3121 | ttctatgcaa | gccttgctag | acagccaggt | tagcctttgc | cctgtcaccc | ccgaatcatg |
| 3181 | acccacccag | tgtctttcga | ggtgggtttg | taccttcctt | aagccaggaa | agggattcat |
| 3241 | ggcgtcggaa | atgatctggc | tgaatccgtg | gtggcaccga | gaccaaactc | attcaccaaa |
| 3301 | tgatgccact | tcccagaggc | agagcctgag | tcactggtca | cccttaatat | ttattaagtg |
| 3361 | cctgagacac | ccggttacct | tggccgtgag | gacacgtggc | ctgcacccag | gtgtggctgt |
| 3421 | caggacacca | gcctggtgcc | catcctcccg | acccctaccc | acttccattc | ccgtggtctc |
| 3481 | cttgcacttt | ctcagttcag | agttgtacac | tgtgtacatt | tggcatttgt | gttattattt |
| 3541 | tgcactgttt | tctgtcgtgt | gtgttgggat | gggatcccag | gccagggaaa | gcccgtgtca |
| 3601 | atgaatgccg | gggacagaga | ggggcaggtt | gaccgggact | tcaaagccgt | gatcgtgaat |
| 3661 | atcgagaact | gccattgtcg | tctttatgtc | cgcccaccta | gtgcttccac | ttctatgcaa |
| 3721 | atgcctccaa | gccattcact | tccccaatct | tgtcgttgat | gggtatgtgt | ttaaaacatg |
| 3781 | cacggtgagg | ccgggcgcag | tggctcacgc | ctgtaatccc | agcactttgg | gaggccgagg |
| 3841 | cgggtggatc | atgaggtcag | gagatcgaga | ccatcctggc | taacacgtga | aaccccgtct |
| 3901 | ctactaaaaa | tacaaaaaat | tagccgggcg | tggtggcggg | cacctgtagt | cccagctact |
| 3961 | cgggaggctg | aggcaggaga | atggtgtgaa | cccgggaagc | ggagcttgca | gtgagccgag |
| 4021 | attgcgccac | tgcagtccgc | agtctggcct | gggcgacaga | gcgagactcc | gtctcaaaaa |
| 4081 | aaaaaaacaa | aaaaaaacca | tgcatggtgc | atcagcagcc | catggcctct | ggccaggcat |
| 4141 | ggcgaggctg | aggtgggagg | atggtttgag | ctcaggcatt | tgaggctgtc | gtgagctatg |
| 4201 | attatgccac | tgctttccag | cctgggcaac | atagtaagac | cccatctctt | aaaaaatgaa |
| 4261 | tttggccaga | cacaggtgcc | tcacgcctgt | aatcccagca | ctttgggagg | ctgagctgga |
| 4321 | tcacttgagt | tcaggagttg | gagaccaggc | ctgagcaaca | aagcgagatc | ccatctctac |
| 4381 | aaaaaccaaa | aagttaaaaa | tcagctgggt | acggtggcac | gtgcctgtga | tcccagctac |
| 4441 | ttgggaggct | gaggcaggag | gatcgcctga | gcccaggagg | tggaggttgc | agtgagccat |
| 4501 | gatcgagcca | ctgcactcca | gcctgggcaa | cagatgaaga | ccctatttca | gaaatacaac |
| 4561 | tataaaaaaa | taaataaatc | ctccagtctg | gatcgtttga | cgggacttca | ggttctttct |
| 4621 | gaaatcgccg | tgttactgtt | gcactgatgt | ccggagagac | agtgacagcc | tccgtcagac |
| 4681 | tcccgcgtga | agatgtcaca | agggattggc | aattgtcccc | agggacaaaa | cactgtgtcc |
| 4741 | cccccagtgc | agggaaccgt | gataagcctt | tctggtttcg | gagcacgtaa | atgcgtccct |
| 4801 | gtacagatag | tggggatttt | ttgttatgtt | tgcactttgt | atattggttg | aaactgttat |
| 4861 | cacttatata | tatatatata | cacacatata | tataaaatct | atttattttt | gcaaaccctg |
| 4921 | gttgctgtat | ttgttcagtg | actattctcg | gggccctgtg | tagggggtta | ttgcctctga |
| 4981 | aatgcctctt | ctttatgtac | aaagattatt | tgcacgaact | ggactgtgtg | caacgctttt |
| 5041 | tgggagaatg | atgtccccgt | tgtatgtatg | agtggcttct | gggagatggg | tgtcactttt |
| 5101 | taaaccactg | tatagaaggt | ttttgtagcc | tgaatgtctt | actgtgatca | |
| 5161 | ttaaatgaac | caa | | | | |

In a particular embodiment, the nucleotide sequence encoding LDL-R comprises nucleotides 87-2669 of SEQ ID NO: 3. In certain embodiments, the inhibitory nucleic acid molecule targets a region within nucleotides 87-2669 of SEQ ID NO: 3.

An example of an amino acid sequence of LDL-R is (SEQ ID NO: 4):

| | | | | | | |
|---|---|---|---|---|---|---|
| 1 | MGPWGWKLRW | TVALLLAAAG | TAVGDRCERN | EFQCQDGKCI | SYKWVCDGSA | ECQDGSDESQ |
| 61 | ETCLSVTCKS | GDFSCGGRVN | RCIPQFWRCD | GQVDCDNGSD | EQGCPPKTCS | QDEFRCHDGK |
| 121 | CISRQFVCDS | DRDCLDGSDE | ASCPVLTCGP | ASFQCNSSTC | IPQLWACDND | PDCEDGSDEW |
| 181 | PQRCRGLYVF | QGDSSPCSAF | EFHCLSGECI | HSSWRCDGGP | DCKDKSDEEN | CAVATCRPDE |
| 241 | FQCSDGNCIH | GSRQCDREYD | CKDMSDEVGC | VNVTLCEGPN | KFKCHSGECI | TLDKVCNMAR |
| 301 | DCRDWSDEPI | KECGTNECLD | NNGGCSHVCN | DLKIGYECLC | PDGFQLVAQR | RCEDIDECQD |
| 361 | PDTCSQLCVN | LEGGYKCQCE | EGFQLDPHTK | ACKAVGSIAY | LFFTNRHEVR | KMTLDRSEYT |
| 421 | SLIPNLRNVV | ALDTEVASNR | IYWSDLSQRM | ICSTQLDRAH | GVSSYDTVIS | RDIQAPDGLA |
| 481 | VDWIHSNIYW | TDSVLGTVSV | ADTKGVKRKT | LFRENGSKPR | AIVVDPVHGF | MYWTDWGTPA |
| 541 | KIKKGGLNGV | DIYSLVTENI | QWPNGITLDL | LSGRLYWVDS | KLHSISSIDV | NGGNRKTILE |
| 601 | DEKRLAHPFS | LAVFEDKVEW | TDIINEAIFS | ANRLTGSDVN | LLAENLLSPE | DMVLFHNLTQ |
| 661 | PRGVNWCERT | TLSNGGCQYL | CLPAPQINPH | SPKFTCACPD | GMLLARDMRS | CLTEAEAAVA |
| 721 | TQETSTVRLK | VSSTAVRTQH | TTTRPVPDTS | RLPGATPGLT | TVEIVTMSHQ | ALGDVAGRGN |
| 781 | EKKPSSVRAL | SIVLPIVLLV | FLCLGVFLLW | KNWRLKNINS | INFDNPVYQK | TTEDEVHICH |
| 841 | NQDGYSYPSR | QMVSLEDDVA | | | | |

In a particular embodiment, the LDL-R is the mature form. In a particular embodiment, the LDL-R lacks the 21 amino acid N-terminus signal peptide. In a particular embodiment, the LDL-R has at least 80%, 85%, 90%, 95%, 97%, 99%, or 100% identity, particularly at least 97%, or 99% identity, with one of the above sequences.

In accordance with another aspect of the present invention, methods for reducing the elimination of viral vectors in vivo are provided. In a particular embodiment, the method comprises the addition of CD47 on the surface of the viral vector. In a particular embodiment, the viral vector is a gene therapy vector. In a particular embodiment, the viral vector is one of the viral vectors described herein (e.g., AAV vector or lentiviral vector (e.g., VSV-G pseudotyped vector)). In a particular embodiment, CD47 is expressed or overexpressed in the packaging cell lines for the viral vector, thereby resulting in the inclusion of CD47 on the viral particles.

CD47 is a membrane molecule that blocks elimination by macrophages of the immune system. In a particular embodiment, the CD47 is human. In a particular embodiment, the CD47 is the mature form of the protein. Examples of amino acid and nucleotide sequences of CD47 are provided in Gene ID: 961 and GenBank Accession Nos: NM_001777.4 and NP_001768.1. An example of a nucleotide sequence encoding CD47 is (SEQ ID NO: 5):

| | | | | | | |
|---|---|---|---|---|---|---|
| 1 | gcagcctggg | cagtgggtcc | tgcctgtgac | gcgcggcggc | ggtcggtcct | gcctgtaacg |
| 61 | gcggcggcgg | ctgctgctcc | ggacacctgc | ggcggcggcg | gcgaccccgc | ggcgggcgcg |
| 121 | gagatgtggc | ccctggtagc | ggcgctgttg | ctgggctcgg | cgtgctgcgg | atcagctcag |
| 181 | ctactattta | ataaaacaaa | atctgtagaa | ttcacgtttt | gtaatgacac | tgtcgtcatt |
| 241 | ccatgctttg | ttactaatat | ggaggcacaa | aacactactg | aagtatacgt | aaagtggaaa |
| 301 | tttaaaggaa | gagatattta | cacctttgat | ggagctctaa | acaagtccac | tgtccccact |
| 361 | gactttagta | gtgcaaaaat | tgaagtctca | caattactaa | aaggagatgc | ctctttgaag |
| 421 | atggataaga | gtgatgctgt | ctcacacaca | ggaaactaca | cttgtgaagt | aacagaatta |
| 481 | accagagaag | gtgaaacgat | catcgagcta | aaatatcgtg | ttgtttcatg | gttttctcca |
| 541 | aatgaaaata | ttcttattgt | tattttccca | atttttgcta | tactcctgtt | ctggggacag |
| 601 | tttggtatta | aaacacttaa | atatagatcc | ggtggtatgg | atgagaaaac | aattgcttta |
| 661 | cttgttgctg | gactagtgat | cactgtcatt | gtcattgttg | gagccattct | tttcgtccca |
| 721 | ggtgaatatt | cattaaagaa | tgctactggc | cttggtttaa | ttgtgacttc | tacagggata |
| 781 | ttaatattac | ttcactacta | tgtgtttagt | acagcgattg | gattaacctc | cttcgtcatt |
| 841 | gccatattgg | ttattcaggt | gatagcctat | atcctcgctg | tggttggact | gagtctctgt |
| 901 | attgcggcgt | gtataccaat | gcatggccct | cttctgattt | caggtttgag | tatcttagct |
| 961 | ctagcacaat | tacttggact | agtttatatg | aaatttgtgg | cttccaatca | gaagactata |
| 1021 | caacctccta | ggaaagctgt | agaggaaccc | cttaatgcat | tcaaagaatc | aaaaggaatg |
| 1081 | atgaatgatg | aataactgaa | gtgaagtgat | ggactccgat | ttggagagta | gtaagacgtg |
| 1141 | aaaggaatac | acttgtgttt | aagcaccatg | gccttgatga | ttcactgttg | gggagaagaa |
| 1201 | acaagaaaag | taactggttg | tcacctatga | gacccttacg | tgattgttag | ttaagttttt |
| 1261 | attcaaagca | gctgtaattt | agttaataaa | ataattatga | tctatgttgt | ttgcccaatt |
| 1321 | gagatccagt | tttttgttgt | tatttttaat | caattagggg | caatagtaga | atggacaatt |
| 1381 | tccaagaatg | atgcctttca | ggtcctaggg | cctctggcct | ctaggtaacc | agtttaaatt |
| 1441 | ggttcagggt | gataactact | tagcactgcc | ctggtgatta | cccagagata | tctatgaaaa |
| 1501 | ccagtggctt | ccatcaaacc | tttgccaact | caggttcaca | gcagctttgg | gcagttatgg |
| 1561 | cagtatggca | ttagctgaga | ggtgtctgcc | acttctgggt | caatggaata | ataaattaag |
| 1621 | tacaggcagg | aatttggttg | ggagcatctt | gtatgatctc | cgtatgatgt | gatattgatg |
| 1681 | gagatagtgg | tcctcattct | tgggggttgc | cattcccaca | ttcccccttc | aacaaacagt |
| 1741 | gtaacaggtc | cttcccagat | ttagggtact | tttattgatg | gatatgtttt | ccttttattc |
| 1801 | acataacccc | ttgaaaccct | gtcttgtcct | cctgttactt | gcttctgctg | tacaagatgt |
| 1861 | agcacctttt | ctcctctttg | aacatggtct | agtgacacgg | tagcaccagt | tgcaggaagg |
| 1921 | agccagactt | gttctcagag | cactgtgttc | acacttttca | gcaaaaatag | ctatggttgt |
| 1981 | aacatatgta | ttcccttcct | ctgatttgaa | ggcaaaaatc | tacagtgttt | cttcacttct |
| 2041 | tttctgatct | ggggcatgaa | aaaagcaaga | ttgaaatttg | aactatgagt | ctcctgcatg |
| 2101 | gcaacaaaat | gtgtgtcacc | atcaggccaa | caggccagcc | cttgaatggg | gatttattac |
| 2161 | tcttgtatct | atgttgcatg | ataaacattc | atcaccttcc | tcctgtagtc | ctgcctcgta |
| 2221 | ctccccttcc | cctatgattg | aaaagtaaac | aaaacccaca | tttcctatcc | tggttagaag |
| 2281 | aaaattaatg | ttctgacagt | tytgatcgcc | tggagtactt | ttagactttt | agcattcgtt |
| 2341 | ttttacctgt | ttgtggatgt | gtgtttgtat | gtgcatacgt | atgagatagg | cacatgcatc |
| 2401 | ttctgtatgg | acaaaggtgg | ggtacctaca | ggagagcaaa | ggttaatttt | gtgcttttag |
| 2461 | taaaaacatt | taaatacaaa | gttctttatt | gggtggaatt | atatttgatg | caaatatttg |
| 2521 | atcacttaaa | acttttaaaa | cttctaggta | atttgccacg | ctttttgact | gctcaccaat |
| 2581 | accctgtaaa | aatacgtaat | tcttcctgtt | tgtgtaataa | gatattcata | tttgtagttg |
| 2641 | cattaataat | agttatttct | tagtccatca | gatgttcccg | tgtgcctctt | ttatgccaaa |
| 2701 | ttgattgtca | tatttcatgt | tgggaccaag | tagtttgccc | atggcaaacc | taaatttatg |
| 2761 | acctgctgag | gcctctcaga | aaactgagca | tactagcaag | acagctcttc | ttgaaaaaaa |
| 2821 | aaatatgtat | acacaaatat | atacgtatat | ctatatatac | gtatgtatat | acacacatgt |
| 2881 | atattcttcc | ttgattgtgt | agctgtccaa | aataataaca | tatatagagg | gagctgtatt |
| 2941 | cctttataca | aatctgatgg | ctcctgcagc | actttttcct | tctgaaaata | tttacatttt |
| 3001 | gctaacctag | tttgttactt | taaaaatcag | ttttgatgaa | aggagggaaa | agcagatgga |
| 3061 | cttgaaaaag | atccaagctc | ctattagaaa | aggtatgaaa | atctttatag | taaaattttt |
| 3121 | tataaactaa | agttgtacct | tttaatatgt | agtaaactct | catttatttg | gggttcgctc |
| 3181 | ttggatctca | tccatccatt | gtgttctctt | taatgctgcc | tgccttttga | ggcattcact |
| 3241 | gccctagaca | atgccaccag | agatagtggg | ggaaatgcca | gatgaaacca | actcttgctc |
| 3301 | tcactagttg | tcagcttctc | tggataagtg | accacagaag | caggagtcct | cctgcttggg |
| 3361 | catcattggg | ccagttcctt | ctctttaaat | cagatttgta | atggctccca | aattccatca |
| 3421 | catcacattt | aaattgcaga | cagtgttttg | cacatcatgt | atctgttttg | tcccataata |
| 3481 | tgctttttac | tccctgatcc | cagtttctgc | tgttgactct | tccattcagt | tttatttatt |
| 3541 | gtgtgttctc | acagtgacac | catttgtcct | tttctgcaac | aacctttcca | gctacttttg |
| 3601 | ccaaattcta | tttgtcttct | ccttcaaaac | attctccttt | gcagttcctc | ttcatctgtg |
| 3661 | tagctgctct | tttgtctctt | aacttaccat | tcctatagta | ctttatgcat | ctctgcttag |
| 3721 | ttctattagt | tttttggcct | tgctcttctc | cttgatttta | aaattccttc | tatagctaga |
| 3781 | gcttttcttt | ctttcattct | ctcttcctgc | agtgttttgc | atacatcaga | agctaggtac |
| 3841 | ataagttaaa | tgattgagag | ttggctgtat | ttagatttat | cactttttaa | tagggtgagc |
| 3901 | ttgagagttt | tctttctttc | tgtttttttt | ttttgttttt | tttttttttt | tttttttttt |
| 3961 | tttttttgac | taatttcaca | tgctctaaaa | accttcaaag | gtgattattt | ttctcctgga |
| 4021 | aactccaggt | ccattctgtt | taaatcccta | agaatgtcag | aattaaaata | acagggctat |
| 4081 | cccgtaattg | gaaatatttc | ttttttcagg | atgctatagt | caatttagta | agtgaccacc |
| 4141 | aaattgttat | ttgcactaac | aaagctcaaa | acacgataag | tttactcctc | catctcagta |
| 4201 | ataaaaatta | agctgtaatc | aaccttctag | gtttctcttg | tcttaaaatg | ggtattcaaa |
| 4261 | aatggggatc | tgtggtgtat | gtatggaaac | acatactcct | taatttacct | gttgttggaa |
| 4321 | actggagaaa | tgattgtcgg | gcaaccgttt | attttttatt | gtattttatt | tggttgaggg |
| 4381 | atttttttat | aaacagtttt | acttgtgtca | tattttaaaa | ttactaactg | ccatcacctg |
| 4441 | ctggggtcct | ttgttaggtc | attttcagtg | actaataggg | ataatccagg | taactttgaa |
| 4501 | gagatgagca | gtgagtgacc | aggcagtttt | tctgccttta | gctttgacag | ttcttaatta |
| 4561 | agatcattga | agaccagctt | tctcataaat | ttctcttttt | gaaaaaaaga | aagcatttgt |
| 4621 | actaagctcc | tctgtaagac | aacatcttaa | atcttaaaag | tgttgttatc | atgactggtg |
| 4681 | agagaagaaa | acattttgtt | tttattaaat | ggagcattat | ttacaaaaag | ccattgttga |
| 4741 | gaattagatc | ccacatcgta | taaatatcta | ttaaccattc | taaataaaga | gaactccagt |
| 4801 | gttgctatgt | gcaagatcct | ctcttggagc | ttttttgcat | agcaattaaa | ggtgtgctat |
| 4861 | ttgtcagtag | ccattttttt | gcagtgattt | gaagaccaaa | gttgttttac | agctgtgtta |
| 4921 | ccgttaaagg | tttttttttt | tatatgtatt | aaatcaattt | atcactgttt | aaagctttga |
| 4981 | atatctgcaa | tctttgccaa | ggtacttttt | tatttaaaaa | aaaacataac | tttgtaaata |
| 5041 | ttaccctgta | atattatata | tacttaataa | aacattttaa | gctattttgt | tgggctattt |
| 5101 | ctattgctgc | tacagcagac | cacaagcaca | tttctgaaaa | atttaattta | ttaatgtatt |
| 5161 | tttaagttgc | ttatattcta | ggtaacaatg | taaagaatga | tttaaaatat | taattatgaa |
| 5221 | ttttttgagt | ataataccca | ataagctttt | aattagagca | gagttttaat | taaaagtttt |
| 5281 | aaatcagtcc | aa | | | | |

In a particular embodiment, the nucleotide sequence encoding CD47 comprises nucleotides 124-1095 of SEQ ID NO: 5. In certain embodiments, the inhibitory nucleic acid molecule targets a region within nucleotides 124-1095 of SEQ ID NO: 5.

An example of an amino acid sequence of CD47 is (SEQ ID NO: 6):

| | | | | | | |
|---|---|---|---|---|---|---|
| 1 | MWPLVAALLL | GSACCGSAQL | LENKTKSVEF | TFCNDTVVIP | CFVTNMEAQN | TTEVYVKWKF |
| 61 | KGRDIYTFDG | ALNKSTVPTD | FSSAKIEVSQ | LLKGDASLKM | DKSDAVSHTG | NYTCEVTELT |
| 121 | REGETIIELK | YRVVSWFSPN | ENILIVIFPI | FAILLFWGQF | GIKTLKYRSG | GMDEKTIALL |
| 181 | VAGLVITVIV | IVGAILFVPG | EYSLKNATGL | GLIVTSTGIL | ILLHYYVFST | AIGLTSFVIA |
| 241 | ILVIQVIAYI | LAVVGLSLCI | AACIPMHGPL | LISGLSILAL | AQLLGLVYMK | FVASNQKTIQ |
| 301 | PPRKAVEEPL | NAFKESKGMM | NDE | | | |

In a particular embodiment, the CD47 lacks the 18 amino acid N-terminus signal peptide. In a particular embodiment, the CD47 has at least 80%, 85%, 90%, 95%, 97%, 99%, or 100% identity, particularly at least 97%, or 99% identity, with one of the above sequences.

In accordance with another aspect of the present invention, VSV-G variants are provided. In a particular embodiment, the VSV-G variant targets HSC or other cells (e.g., other than liver cells). The instant invention also encompasses viruses comprising (e.g., pseudotyped with) the VSV-G variant. In a particular embodiment, the VSV-G variant is expressed in the virus packaging cell line. Viruses comprising the VSV-G variants can be used in vivo, in vitro, or ex vivo. While the variants described herein are provided in the context of VSV-G, the variations can be applied to other viral envelope proteins, such as cocal virus envelope glycoprotein which is closely related to VSV-G.

VSV-G is very effective at enabling vector entry and transduction at relatively low titers. However, as LDL-R is ubiquitously expressed in the body, VSV-G guided vectors lack tissue specificity and promiscuously transduce many cell types, significantly hampering any attempt to therapeutically transduce a specific tissue or cell type. Therefore, modifications to VSV-G itself that allow for the retargeting of VSV-G to specific cell types and specific receptors apart from LDL-R, while retaining most of VSV-G's fusion efficiency, are desirable to facilitate in vivo gene therapies. Even modifications that do not completely abrogate VSV-G's native LDL-R targeting are advantageous if the engineered targeting to the receptor of interest is sufficiently high or increased.

In a particular embodiment, the VSV-G comprises a targeting moiety specific for the receptor of interest. In a particular embodiment, the targeting moiety is an antibody or antibody fragment (e.g., scFv), designed ankyrin repeat protein (DARPin) (e.g., Pluckthin et al. (2015) Annu. Rev. Pharmacol. Toxicol., 55:489-511), or a receptor cognate (e.g., a cytokine or receptor-binding fragment thereof). The targeting moiety may be directly covalently attached to the VSV-G or attached by a flexible linker (e.g., a glycine-serine repeat (e.g., (GGGGS)ₓ, wherein x is 1-5 (SEQ ID NO: 7)).

In a particular embodiment, the targeting moiety is attached to the N-terminal region or the N-terminus of the VSV-G. VSV-G is a type-1 protein with an N-terminus exposed extracellularly/extravirally and a C-terminus within the cell or viral particle. In a particular embodiment, the targeting moiety is attached to the C-terminus of the VSV-G, wherein the targeting moiety is attached to the VSV-G via a linker comprising a transmembrane domain (e.g., the transmembrane domain of VSV-G or a G-Protein Coupled Receptors (GPCRs) transmembrane domain). The presence of the transmembrane domain in the linker will allow the targeting moiety at the C-terminus to be extracellular/extraviral. In a particular embodiment, the VSV-G variant further comprises signaling domain(s) from a transmembrane protein such as a multi-pass transmembrane protein such as the G-Protein Coupled Receptors (GPCRs).

In a particular embodiment, the targeting moiety is non-covalently associated with VSV-G. In a particular embodiment, VSV-G and the targeting moiety are expressed as separate proteins, but both would have motifs (e.g., C-terminal motifs) that are reciprocal, allowing for non-covalent association (e.g., cytoplasmic/intervirion association). In a particular embodiment, the motif includes, without limitation, PDZ domain/cognate peptide and DARPin/cognate peptide. Non-covalent association has the advantage of allowing for stoichiometric modulation of VSV-G to targeting moiety, thereby allowing for the fine-tuning of vector activity. Additionally, this technique allows for the utilization of constructs that essentially, for functionally purposes, have two exposed termini (e.g., N-terminal ends or one N and one C-terminal end, etc.), increasing the engineering possibilities. A further advantage of this system is that it is modular in nature, allowing for the targeting of different receptors and thereby cell types by changing the specificity of the targeting motif.

The components (e.g., viruses and/or inhibitors) as described herein will generally be administered to a patient as a pharmaceutical preparation. The term “patient” or “subject” as used herein refers to human or animal subjects. The components of the instant invention may be employed therapeutically, under the guidance of a physician for the treatment of the indicated disease or disorder.

The pharmaceutical preparation comprising the components of the invention may be conveniently formulated for administration with an acceptable medium (e.g., pharmaceutically acceptable carrier) such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the agents in the chosen medium may be varied and the medium may be chosen based on the desired route of administration of the pharmaceutical preparation. Except insofar as any conventional media or agent is incompatible with the agents to be administered, its use in the pharmaceutical preparation is contemplated.

The compositions of the present invention can be administered by any suitable route, for example, by injection (e.g., for local (direct) or systemic administration), oral, pulmonary, topical, nasal or other modes of administration. The composition may be administered by any suitable means, including parenteral, intramuscular, intravenous, intraarterial, intraperitoneal, subcutaneous, topical, inhalatory, transdermal, intrapulmonary, intraareterial, intrarectal, intramuscular, and intranasal administration. In a particular embodiment, the composition is administered directly to the blood stream (e.g., intravenously). In a particular embodiment, the composition is administered directly to the liver. In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., polysorbate 80), anti oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington: The Science and Practice of Pharmacy, 21st edition, Philadelphia, PA. Lippincott Williams & Wilkins. The pharmaceutical composition of the present invention can be prepared, for example, in liquid form, or can be in dried powder form (e.g., lyophilized for later reconstitution).

As used herein, “pharmaceutically acceptable carrier” includes any and all solvents, dispersion media and the like which may be appropriate for the desired route of administration of the pharmaceutical preparation, as exemplified in the preceding paragraph. The use of such media for pharmaceutically active substances is known in the art. Except insofar as any conventional media or agent is incompatible with the molecules to be administered, its use in the pharmaceutical preparation is contemplated.

Pharmaceutical compositions containing a compound of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous. Injectable suspensions may be prepared, in which case appropriate liquid carriers, suspending agents and the like may be employed. Pharmaceutical preparations for injection are known in the art. If injection is selected as a method for administering the therapy, steps should be taken to ensure that sufficient amounts of the molecules reach their target cells to exert a biological effect.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art. The appropriate dosage unit for the administration of the molecules of the instant invention may be determined by evaluating the toxicity of the molecules in animal models. Appropriate dosage unit may also be determined by assessing the efficacy of the treatment in combination with other standard therapies.

The pharmaceutical preparation comprising the molecules of the instant invention may be administered at appropriate intervals, for example, at least twice a day or more until the pathological symptoms are reduced or alleviated, after which the dosage may be reduced to a maintenance level. The appropriate interval in a particular case would normally depend on the condition of the patient.

### Definitions

The singular forms “a,” “an,” and “the” include plural referents unless the context clearly dictates otherwise.

The terms “isolated” is not meant to exclude artificial or synthetic mixtures with other compounds or materials, or the presence of impurities that do not interfere with the fundamental activity, and that may be present, for example, due to incomplete purification, or the addition of stabilizers.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxilliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in Remington: The Science and Practice of Pharmacy, (Lippincott, Williams and Wilkins); Liberman, et al., Eds., Pharmaceutical Dosage Forms, Marcel Decker, New York, N.Y.; and Rowe, et al., Eds., Handbook of Pharmaceutical Excipients, Pharmaceutical Pr.

The term “treat” as used herein refers to any type of treatment that imparts a benefit to a patient suffering from a disease or disorder, including improvement in the condition of the patient (e.g., in one or more symptoms), delay in the progression of the condition, etc.

As used herein, the term “prevent” refers to the prophylactic treatment of a subject who is at risk of developing a condition and/or sustaining a disease or disorder, resulting in a decrease in the probability that the subject will develop conditions associated with the disease.

A “therapeutically effective amount” of a compound or a pharmaceutical composition refers to an amount effective to prevent, inhibit, or treat a particular injury and/or the symptoms thereof. For example, “therapeutically effective amount” may refer to an amount sufficient to modulate the pathology associated with a hemoglobinopathy or thalassemia.

As used herein, the term “subject” refers to an animal, particularly a mammal, particularly a human.

The term “vector” refers to a carrier nucleic acid molecule (e.g., RNA or DNA) into which a nucleic acid sequence can be inserted, e.g., for introduction into a host cell where it may be expressed and/or replicated. Examples of vectors include, without limitation, a plasmid, cosmid, bacmid, phage or virus. A vector may be either RNA or DNA and may be single or double stranded. A vector may comprise expression operons or elements such as, without limitation, transcriptional and translational control sequences, such as promoters, enhancers, translational start signals, polyadenylation signals, terminators, and the like, and which facilitate the expression of a polynucleotide or a polypeptide coding sequence in a host cell or organism. An “expression vector” is a specialized vector that contains a gene or nucleic acid sequence with the necessary operably linked regulatory regions needed for expression in a host cell. The term “operably linked” means that the regulatory sequences necessary for expression of a coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, amino acids, or nucleic acids.

An “antibody” or “antibody molecule” is any immunoglobulin, including antibodies and fragments thereof, that binds to a specific antigen. As used herein, antibody or antibody molecule contemplates intact immunoglobulin molecules, immunologically active portions/fragment (e.g., antigen binding portion/fragment) of an immunoglobulin molecule, and fusions of immunologically active portions of an immunoglobulin molecule. Antibody fragments include, without limitation, immunoglobulin fragments including, without limitation: single domain (Dab; e.g., single variable light or heavy chain domain), Fab, Fab′, F(ab′)₂, and F(v); and fusions (e.g., via a linker) of these immunoglobulin fragments including, without limitation: scFv, scFv₂, scFv-Fc, minibody, diabody, triabody, and tetrabody.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

The phrase “small, interfering RNA (siRNA)” refers to a short (typically less than 30 nucleotides long, particularly 12-30 or 20-25 nucleotides in length) double stranded RNA molecule. Typically, the siRNA modulates the expression of a gene to which the siRNA is targeted. Methods of identifying and synthesizing siRNA molecules are known in the art (see, e.g., Ausubel et al., Current Protocols in Molecular Biology, John Wiley and Sons, Inc). Short hairpin RNA molecules (shRNA) typically consist of short complementary sequences (e.g., an siRNA) separated by a small loop sequence (e.g., 6-15 nucleotides, particularly 7-10 nucleotides) wherein one of the sequences is complimentary to the gene target. shRNA molecules are typically processed into an siRNA within the cell by endonucleases. Exemplary modifications to siRNA molecules are provided in U.S. Application Publication No. 20050032733. For example, siRNA and shRNA molecules may be modified with nuclease resistant modifications (e.g., phosphorothioates, locked nucleic acids (LNA), 2′-O-methyl modifications, or morpholino linkages). Expression vectors for the expression of siRNA or shRNA molecules may employ a strong promoter which may be constitutive or regulated. Such promoters are well known in the art and include, but are not limited to, RNA polymerase II promoters, the T7 RNA polymerase promoter, and the RNA polymerase III promoters U6 and H1.

“Antisense nucleic acid molecules” or “antisense oligonucleotides” include nucleic acid molecules (e.g., single stranded molecules) which are targeted (complementary) to a chosen sequence (e.g., to translation initiation sites and/or splice sites) to inhibit the expression of a protein of interest. Such antisense molecules are typically between about 15 and about 50 nucleotides in length, more particularly between about 15 and about 30 nucleotides, and often span the translational start site of mRNA molecules. Antisense constructs may also be generated which contain the entire sequence of the target nucleic acid molecule in reverse orientation. Antisense oligonucleotides targeted to any known nucleotide sequence can be prepared by oligonucleotide synthesis according to standard methods. Antisense oligonucleotides may be modified as described above to comprise nuclease resistant modifications. In certain embodiments, antisense oligonucleotides target regions of the mRNA which do not comprise secondary and tertiary structures. in certain embodiments, the antisense oligonucleotide may target the 5′ cap, the initiation codon, or the 3′ untranslated region or polyA tail.

“microRNA” or “miRNA” refers to a non-coding single-stranded RNA molecule. Typically, miRNA are less than 30 nucleotides long, particularly 12-30 or 20-25 nucleotides in length.

“Linker” refers to a chemical moiety comprising a covalent bond or a chain of atoms that covalently attach at least two compounds. The linker can be linked to any synthetically feasible position of the compounds, but preferably in such a manner as to avoid blocking the compounds desired activity. Linkers are generally known in the art. In a particular embodiment, the linker may contain from 0 (i.e., a bond) to about 50 atoms, from 0 to about 10 atoms, or from about 1 to about 5 atoms. In a particular embodiment, the linker comprises amino acids, particularly about 1 to about 100 amino acids, about 1 to about 50 amino acids, about 1 to about 25 amino acids, about 1 to about 20 amino acids, about 1 to about 15 amino acids, about 1 to about 10 amino acids, or about 1 to about 5 amino acids.

As used herein “gene therapy” refers to methods where a vector (e.g., an AAV vector or a VSV-G pseudotyped vector) carrying a therapeutic nucleic acid or gene is administered to a cell (e.g., ex vivo) or directly administered to the subject (e.g., in vivo).

The following example is provided to illustrate various embodiments of the present invention. It is not intended to limit the invention in any way.

### Example

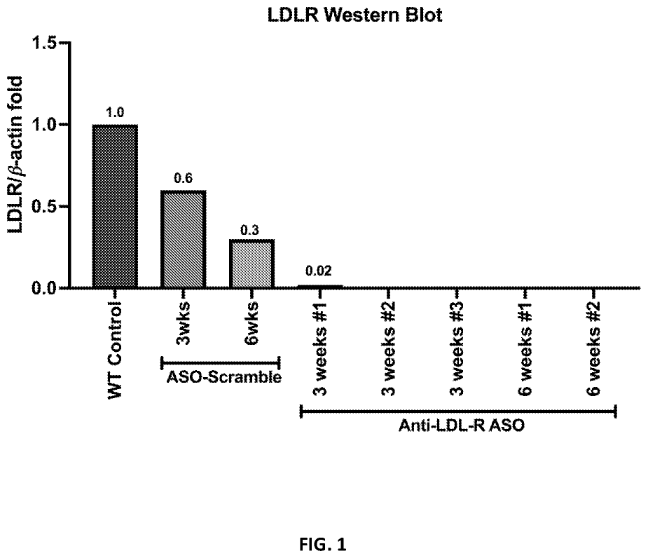

Mice were injected Intraperitoneally twice a week with ASO. At either 3-weeks or 6-weeks of administration mice were sacrificed and their organs collected then snap frozen for storage. For analysis of LDLR levels in the liver, liver sections were thawed, then homogenized in RIPA buffer with a protease inhibitor. The homogenized liver samples were analyzed via western blot utilizing antibodies specific to the LDLR and to beta-actin. The ratio of beta-actin to LDLR was utilized to assess levels of LDLR between samples. As seen in FIG. 1, anti-LDLR antisense oligonucleotides dramatically reduced and effectively eliminated expression of LDLR in the liver.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for increasing adeno-associated virus (AAV) vector gene transfer to cells other than the liver and/or decreasing AAV liver toxicity, said method comprising reducing expression and/or blocking adeno-associated virus receptor (AAVR) in the liver.

2. The method of claim 1, wherein said method comprises administering an AAVR inhibitor to a subject.

3. The method of claim 2, wherein said AAVR inhibitor is an inhibitory nucleic acid molecule or a nucleic acid molecule encoding the inhibitory nucleic acid molecule.

4. The method of claim 3, wherein said inhibitory nucleic acid molecule is an antisense oligonucleotide, siRNA, miRNA, or shRNA.

5. The method of claim 2, wherein said AAVR inhibitor is administered prior to and/or at the same time as an AAV vector.

6. The method of claim 2, wherein said AAVR inhibitor is administered prior to and/or at the same time as an AAV gene therapy vector.

7. The method of claim 1, further comprising administering a hematopoietic stem cell (HSC) mobilization agent to the subject.

8. The method of claim 7, wherein said HSC mobilization agent is plerixafor.

9. The method of claim 1, wherein said AAV vector comprises CD47.

10. A methods for increasing vesicular stomatitis virus G (VSV-G) vector gene transfer to cells other than the liver and/or decreasing liver toxicity, said method comprising reducing expression and/or blocking low-density lipoprotein receptor (LDL-R) in the liver.

11. The method of claim 10, wherein said method comprises administering an LDL-R inhibitor to a subject.

12. The method of claim 11, wherein said LDL-R inhibitor is an inhibitory nucleic acid molecule or a nucleic acid molecule encoding the inhibitory nucleic acid molecule.

13. The method of claim 12, wherein said inhibitory nucleic acid molecule is an antisense oligonucleotide, siRNA, miRNA, or shRNA.

14. The method of claim 10, wherein said LDL-R inhibitor is administered prior to and/or at the same time as an VSV-G vector.

15. The method of claim 10, wherein said LDL-R inhibitor is administered prior to and/or at the same time as a VSV-G gene therapy vector.

16. The method of claim 10, further comprising administering a hematopoietic stem cells (HSC) mobilization agent to the subject.

17. The method of claim 16, wherein said HSC mobilization agent is plerixafor.

18. The method of claim 10, wherein said VSV-G vector comprises CD47.


SEQUENCE LISTING (7 sequences)

SEQ ID NO: 1 - DNA, 4777 bp, Homo sapiens
gtttccggccgccgtcgctgtccagggaggctgaggcgagaggtagctgtccgggtggggagcccgcactaccttcttcctcttcctcctcctcctccgggtgaggggagcgaaggttgggggtccccgagcccatggaccaggaggaggcggaggccgccgagagccggggccccgctatgtggccctgagccccgtgtactggttctgcctgtctggagggccatggagaagaggctgggagtcaagccaaatcctgcttcctggattttatcaggatattattggcagacatctgcgaagtggttgagaagcctgtacctgttttatacttgcttttgcttcagcgttctgtggttgtcaacagatgccagtgagagcaggtgccagcaggggaagacacaatttggagttggcctgagatctgggggagaaaatcacctctggcttcttgaaggaaccccctctctccagtcatgttgggctgcctgctgccaggactctgcctgccatgtcttttggtggctagaagggatgtgcattcaggcagactgcagcaggccccagagctgccgggcttttaggacacactcctccaattccatgctggtgtttttaaaaaaattccaaactgcagatgatttgggctttctacctgaagatgatgtaccacatcttctggggctaggttggaactgggcatcttggaggcagagcccacccagagctgcactcagacctgctgtatcttccagtgaccagcagagcttaatcaggaagcttcagaagagaggtagtcccagtgacgtagttacacctatagtgacacagcattctaaagtgaatgactccaacgaattaggtggtctgactaccagtggctctgcagaggtccacaaggcgattacaatttccagtcccctaaccacagacctgactgcagagctgtctggtgggccaaagaatgtatcagtgcaacctgaaatatcagagggtcttgctactacgcccagcactcaacaagtaaaaagttctgagaaaacccagattgctgtcccccagccagtggctccctcctacagttatgctacccctaccccccaggcctctttccagagcacctcagcaccatacccagttataaaggaactggtggtatctgctggagagagtgtccagataaccctgcctaagaatgaagttcaattaaatgcatatgttctccaagaaccacctaaaggagaaacctacacctacgactggcagctgattactcatcctagagactacagtggagaaatggaagggaaacattcccagatcctcaaactatcgaagctcactccaggcctgtatgaattcaaagtgattgtagagggtcaaaatgcccatggggaaggctatgtgaacgtgacagtcaagccagagccccgtaagaatcggccccccattgctattgtgtcacctcagttccaggagatctctttgccaaccacttctacagtcattgatggcagtcaaagcactgatgatgataaaatcgttcagtaccattgggaagaacttaaggggcctctaagagaagagaagatttctgaagatacagccatattaaaactaagtaaactcgtccctgggaactacactttcagcttgactgtagtagactctgatggagctaccaactctactactgcaaacctgacagtgaacaaagctgtggattacccccctgtggccaacgcaggccccaaccaagtgatcaccctgccccaaaactccatcaccctctttgggaaccagagcactgatgatcatggcatcaccagctatgagtggtcactcagcccaagcagcaaagggaaagtggtggagatgcagggtgttagaacaccaaccttacagctctctgcgatgcaagaaggagactacacttaccagctcacagtgactgacacaataggacagcaggccactgctcaagtgactgttattgtgcaacctgaaaacaataagcctcctcaggcagatgcaggcccagataaagagctgacccttcctgtggatagcacaaccctggatggcagcaagagctcagatgatcagaaaattatctcatatctctgggaaaaaacacagggacctgatggggtgcagctcgagaatgctaacagcagtgttgctactgtgactgggctgcaagtggggacctatgtgttcaccttgactgtcaaagatgagaggaacctgcaaagccagagctctgtgaatgtcattgtcaaagaagaaataaacaaaccacctatagccaagataactgggaatgtggtgattaccctacccacgagcacagcagagctggatggctctaagtcctcagatgacaagggaatagtcagctacctctggactcgagatgaggggagcccagcagcaggggaggtgttaaatcactctgaccatcaccctatcctttttctttcaaacctggttgagggaacctacacttttcacctgaaagtgaccgatgcaaagggtgagagtgacacagaccggaccactgtggaggtgaaacctgatcccaggaaaaacaacctggtggagatcatcttggatatcaacgtcagtcagctaactgagaggctgaaggggatgttcatccgccagattggggtcctcctgggggtgctggattccgacatcattgtgcaaaagattcagccgtacacggagcagagcaccaaaatggtattttttgttcaaaacgagcctccccaccagatcttcaaaggccatgaggtggcagcgatgctcaagagtgagctgcggaagcaaaaggcagactttttgatattcagagccttggaagtcaacactgtcacatgtcagctgaactgttccgaccatggccactgtgactcgttcaccaaacgctgtatctgtgaccctttttggatggagaatttcatcaaggtgcagctgagggatggagacagcaactgtgagtggagcgtgttatatgttatcattgctacctttgtcattgttgttgccttgggaatcctgtcttggactgtgatctgttgttgtaagaggcaaaaaggaaaacccaagaggaaaagcaagtacaagatcctggatgccacggatcaggaaagcctggagctgaagccaacctcccgagcaggcatcaaacagaaaggccttttgctaagtagcagcctgatgcactccgagtcagagctggacagcgatgatgccatctttacatggccagaccgagagaagggcaaactcctgcatggtcagaatggctctgtacccaacgggcagacccctctgaaggccaggagcccgcgggaggagatcctgtagccacctggtctgtctcctcagggcagggcccagcacactgcccggccagtcctcctacctcccgagtctgcgggcagctgctgtcccagcatctgctggtcatttcgccctgacagtcccaaccagaacccctgggacttgaatccagagacgtcctccaggaacccctcaacgaagctgtgaatgaagaggtttcctctttaaacctgtctggtgggcccccagatatcctcacctcagggcctcctttttttgcaaactcctcccctcccccgagggcagacccagccagctgctaagctctgcagctccccagtggacagtgtcattgtgcccagagtgctgcaaggtgaggcctgctgtgctgcccgcacacctgagtgcaaaaccaagcactgtgggcatggtgtttccctctctggggtagagtacgccctctcgctgggcaaagaggaagtggcacccctcccctcaccacagatgctgagatggtagcatagaaatgatggccgggcgcggtggctcacgcctgtaatcccagcactttgggaggccgaggcgggcggatcatgaggtcaggagatcaagaccaccctggctaacacggtgaaaccccatctctactaaaaataaaaaaaaaaattagccgggtttggtggcgtatgcctgtaatcccagctactcgggaggctgaggcaggagaattgcttaaacctgggaggtggaggctgcagtgagccaagatcgtgccactgcactccagcctgagtgacagagcaagactccgtcaaaaaaaaaaaaaaaaaaaaagaaatgatatctggcccccccttaacactggagccccactcccttctcccatccggcccgagattagggaggattgactgtgtcagggatggcgggtggcctctctcgctgccagggcccttgtcagagcagccaggctggacagacggcctccctcctctccatctgaccggcacctgctgcttcggggcttaggccaccgctccctgtccccagaggagatagccccagatggactggaatgttgtggcatgagagcgcatgtgtgcgatggccccgctgtggtcccctctctgtccctccatctgtatgtgttctgtgtcccttgcatgtgtgcgtgttagagtgagcgcgtatgcatcaactcattgggctcttggctgctcacaaggcaaatttgacttggaaagactttcatctccttggaaccaagacttcctgagtccccctcaccctggccctgttccaccatggttatctgggtattggggaatggaaactttgggggagtgactttttaaagagacacttataatttctactactgcactactgtccattgtgggatgattaaacatggtatttaactgtg

SEQ ID NO: 2 - protein, 1049 aa, Homo sapiens
MEKRLGVKPNPASWILSGYYWQTSAKWLRSLYLFYTCFCFSVLWLSTDASESRCQQGKTQFGVGLRSGGENHLWLLEGTPSLQSCWAACCQDSACHVFWWLEGMCIQADCSRPQSCRAFRTHSSNSMLVFLKKFQTADDLGFLPEDDVPHLLGLGWNWASWRQSPPRAALRPAVSSSDQQSLIRKLQKRGSPSDVVTPIVTQHSKVNDSNELGGLTTSGSAEVHKAITISSPLTTDLTAELSGGPKNVSVQPEISEGLATTPSTQQVKSSEKTQIAVPQPVAPSYSYATPTPQASFQSTSAPYPVIKELVVSAGESVQITLPKNEVQLNAYVLQEPPKGETYTYDWQLITHPRDYSGEMEGKHSQILKLSKLTPGLYEFKVIVEGQNAHGEGYVNVTVKPEPRKNRPPIAIVSPQFQEISLPTTSTVIDGSQSTDDDKIVQYHWEELKGPLREEKISEDTAILKLSKLVPGNYTFSLTVVDSDGATNSTTANLTVNKAVDYPPVANAGPNQVITLPQNSITLFGNQSTDDHGITSYEWSLSPSSKGKVVEMQGVRTPTLQLSAMQEGDYTYQLTVTDTIGQQATAQVTVIVQPENNKPPQADAGPDKELTLPVDSTTLDGSKSSDDQKIISYLWEKTQGPDGVQLENANSSVATVTGLQVGTYVFTLTVKDERNLQSQSSVNVIVKEEINKPPIAKITGNVVITLPTSTAELDGSKSSDDKGIVSYLWTRDEGSPAAGEVLNHSDHHPILFLSNLVEGTYTFHLKVTDAKGESDTDRTTVEVKPDPRKNNLVEIILDINVSQLTERLKGMFIRQIGVLLGVLDSDIIVQKIQPYTEQSTKMVFFVQNEPPHQIFKGHEVAAMLKSELRKQKADFLIFRALEVNTVTCQLNCSDHGHCDSFTKRCICDPFWMENFIKVQLRDGDSNCEWSVLYVIIATFVIVVALGILSWTVICCCKRQKGKPKRKSKYKILDATDQESLELKPTSRAGIKQKGLLLSSSLMHSESELDSDDAIFTWPDREKGKLLHGQNGSVPNGQTPLKARSPREEIL

SEQ ID NO: 3 - DNA, 5173 bp, Homo sapiens
gtgcaatcgcgggaagccagggtttccagctaggacacagcaggtcgtgatccgggtcgggacactgcctggcagaggctgcgagcatggggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccgacctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaacatctgcctggagtcccgtccctgcccagaacccttcctgagacctcgccggccttgttttattcaaagacagagaagaccaaagcattgcctgccagagctttgttttatatatttattcatctgggaggcagaacaggcttcggacagtgcccatgcaatggcttgggttgggattttggtttcttcctttcctcgtgaaggataagagaaacaggcccggggggaccaggatgacacctccatttctctccaggaagttttgagtttctctccaccgtgacacaatcctcaaacatggaagatgaaaggggaggggatgtcaggcccagagaagcaagtggctttcaacacacaacagcagatggcaccaacgggaccccctggccctgcctcatccaccaatctctaagccaaacccctaaactcaggagtcaacgtgtttacctcttctatgcaagccttgctagacagccaggttagcctttgccctgtcacccccgaatcatgacccacccagtgtctttcgaggtgggtttgtaccttccttaagccaggaaagggattcatggcgtcggaaatgatctggctgaatccgtggtggcaccgagaccaaactcattcaccaaatgatgccacttcccagaggcagagcctgagtcactggtcacccttaatatttattaagtgcctgagacacccggttaccttggccgtgaggacacgtggcctgcacccaggtgtggctgtcaggacaccagcctggtgcccatcctcccgacccctacccacttccattcccgtggtctccttgcactttctcagttcagagttgtacactgtgtacatttggcatttgtgttattattttgcactgttttctgtcgtgtgtgttgggatgggatcccaggccagggaaagcccgtgtcaatgaatgccggggacagagaggggcaggttgaccgggacttcaaagccgtgatcgtgaatatcgagaactgccattgtcgtctttatgtccgcccacctagtgcttccacttctatgcaaatgcctccaagccattcacttccccaatcttgtcgttgatgggtatgtgtttaaaacatgcacggtgaggccgggcgcagtggctcacgcctgtaatcccagcactttgggaggccgaggcgggtggatcatgaggtcaggagatcgagaccatcctggctaacacgtgaaaccccgtctctactaaaaatacaaaaaattagccgggcgtggtggcgggcacctgtagtcccagctactcgggaggctgaggcaggagaatggtgtgaacccgggaagcggagcttgcagtgagccgagattgcgccactgcagtccgcagtctggcctgggcgacagagcgagactccgtctcaaaaaaaaaaaacaaaaaaaaaccatgcatggtgcatcagcagcccatggcctctggccaggcatggcgaggctgaggtgggaggatggtttgagctcaggcatttgaggctgtcgtgagctatgattatgccactgctttccagcctgggcaacatagtaagaccccatctcttaaaaaatgaatttggccagacacaggtgcctcacgcctgtaatcccagcactttgggaggctgagctggatcacttgagttcaggagttggagaccaggcctgagcaacaaagcgagatcccatctctacaaaaaccaaaaagttaaaaatcagctgggtacggtggcacgtgcctgtgatcccagctacttgggaggctgaggcaggaggatcgcctgagcccaggaggtggaggttgcagtgagccatgatcgagccactgcactccagcctgggcaacagatgaagaccctatttcagaaatacaactataaaaaaataaataaatcctccagtctggatcgtttgacgggacttcaggttctttctgaaatcgccgtgttactgttgcactgatgtccggagagacagtgacagcctccgtcagactcccgcgtgaagatgtcacaagggattggcaattgtccccagggacaaaacactgtgtcccccccagtgcagggaaccgtgataagcctttctggtttcggagcacgtaaatgcgtccctgtacagatagtggggattttttgttatgtttgcactttgtatattggttgaaactgttatcacttatatatatatatatacacacatatatataaaatctatttatttttgcaaaccctggttgctgtatttgttcagtgactattctcggggccctgtgtagggggttattgcctctgaaatgcctcttctttatgtacaaagattatttgcacgaactggactgtgtgcaacgctttttgggagaatgatgtccccgttgtatgtatgagtggcttctgggagatgggtgtcactttttaaaccactgtatagaaggtttttgtagcctgaatgtcttactgtgatcaattaaatttcttaaatgaaccaa

SEQ ID NO: 4 - protein, 860 aa, Homo sapiens [sequence omitted]

SEQ ID NO: 5 - DNA, 5292 bp, Homo sapiens
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 6 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 7 - protein, 25 aa, Artificial Sequence
  Linker
  VARIANT 10, 15, 20, 25: Xaa = absent or Ser
  VARIANT 6-9, 11-14, 16-19, 21-24: Xaa = absent or Gly
GGGGSXXXXXXXXXXXXXXXXXXXX